(19) United States
(12) Patent Application Publication
(10) Pub. No.: US 2015/0282752 A1
(43) Pub. Date: Oct. 8, 2015

(54) SPATIAL POSITIONING SURFACE FOR NEUROLOGICAL ASSESSMENT AND TREATMENT

(71) Applicants: Kurt Roots, Minneapolis, MN (US); Monika Drummond Roots, Minneapolis, MN (US)

(72) Inventors: Kurt Roots, Minneapolis, MN (US); Monika Drummond Roots, Minneapolis, MN (US)

(73) Assignee: COGCUBED CORPORATION, Minneapolis, MN (US)

(21) Appl. No.: 14/747,403

(22) Filed: Jun. 23, 2015

Related U.S. Application Data
(63) Continuation-in-part of application No. 14/734,800, filed on Jun. 9, 2015.
Application No. 14/734,800 is a continuation-in-part of application No. 14/064,094, filed on Oct. 25, 2013.
Application No. 14/064,094 is a continuation-in-part of application No. 13/657,332, filed on Oct. 22, 2012, now Pat. No. 9,014,614.
(60) Provisional application No. 62/015,994, filed on Jun. 23, 2014.
(60) Provisional application No. 62/101,300, filed on Jan. 8, 2015.
(60) Provisional application No. 61/719,280, filed on Oct. 26, 2012.
(60) Provisional application No. 61/549,698, filed on Oct. 20, 2011.
(60) Provisional application No. 61/551,384, filed on Oct. 25, 2011.

(51) Int. Cl. A61B 5/16; A63F 3/00; A61B 5/00

(52) U.S. Cl. CPC A61B 5/168; A61B 5/165; A61B 5/4094; A63F 3/00

(57) ABSTRACT

A game board and method for diagnosing and treating neurological disorders such as attention deficits and/or comorbidities, attention deficit hyperactivity disorder, and/or conditions with attentional impairment such as autism spectrum disorder (ASD), anxiety, depression, and epilepsy The game board has two sets of marks, each set forming a line, wherein a first line is orientated in a first direction parallel with a side of the rectangular game board, and a second line orientated in a second direction diagonal to the first line. Objective cubes are variously positioned on the marks and the player is instructed to hit an objective cube according to whether it displays a particular image. Distractors are employed to assist in diagnosing and treating the disorder.

### RELATED APPLICATIONS

The present application is a nonprovisional of U.S. provisional application No. 62/015,994 filed Jun. 23, 2014, and is a continuation-in-part of U.S. patent application Ser. No. 14/734,800, filed Jun. 9, 2015, which is a nonprovisional of U.S. provisional application No. 62/101,300, filed Jun. 10, 2014; and is a continuation-in-part of U.S. patent application Ser. No. 14/064,094, filed Oct. 25, 2013, which is a nonprovisional of U.S. provisional application No. 61/719,280, filed Oct. 26, 2012; and is a continuation-in-part of U.S. patent application Ser. No. 13/657,332, filed Oct. 22, 2012, now U.S. Pat. No. 9,014,614, which claims the benefit of provisional application Nos. 61/549,698, filed Oct. 20, 2011, and 61/551,384, filed Oct. 25, 2011; the contents of which are hereby incorporated by reference in their entirety

### FIELD OF THE INVENTION

### Background of the Invention

1. Field of the Invention

The invention relates to the field of cognitive disorders including assessing attention deficits and/or comorbidities, treating attention deficit hyperactivity disorder, and/or conditions with attentional impairment such as autism spectrum disorder (ASD), anxiety, depression, and epilepsy.

2. Background of the Art

Assessing and treating attention deficits, comorbidities and/or treating other conditions that have attention deficits have fundamentally been a challenge for parents, teachers, and health care providers. For example, there is no specific test for diagnosing attention deficits. Also, there is no aid for differentiating ADHD from conditions that have attentional deficits as a symptom of the disorder. This represents a challenging situation for educators and health care providers. Therefore, the following conditions can be comorbid with ADHD or they may manifest themselves with attentional deficits separately from the disorder.

Learning or language problems

Autism Spectrum Disorders(ASD)

Anxiety disorders

Mood Disorders

Psychotic Disorders

Seizure disorders

Traumatic brain injuries

Frontal lobe pathology

Vision or hearing problems

Sleep disorders

Dementias

Substance Use Disorders

Gathering as much information as possible about the individual from disparate sources is currently the best way to diagnose and treat an individual's symptoms. For example, current diagnostic aids used in assessing ADHD are subjective, expensive, time intensive, and provide little information about accessory movements in response to a stimulus. These diagnostics aids make it difficult to identify ADHD, comorbidities, and/or other attentional related diagnoses.

Consider the typical diagnostic aids for ADHD which are reviewed by health care providers to help make a clinical diagnosis. Tests such as the Conners' Rating Scale require subjective responses from parents and teachers, making coordination difficult. The computerized Conners' Continuous Performance Test (CPT) provides objective data in regards to inattention and impulsive patterns of response. However, it does not provide data regarding accessory movements such as restlessness, hyperactivity, and other inappropriate movements. In addition, it does not give inter-response data. If the subject taking the exam has a reading disorder, it will impair his or her ability to respond accurately and it will increase response time. The T.O.V.A. is another computer based test used as a diagnostic aid for ADHD, which uses a microswitch to record responses. It does utilize auditory and visual stimuli, which removes the reading level limitation. However, it is unable to measure accessory and/or inter-response movements that may be contributing to reaction time and errors of omission and commission.

Upon properly establishing a diagnosis, it is then important to establish a course of treatment. This is vital in both schools and in the medical field. Attentional deficits greatly limit academic aptitude and long term potential leading to the development of some of the common comorbidities such as anxiety and depression. Addressing these symptoms at a young age are crucial in decreasing these detrimental effects long term.

Medications and talk therapy dominate the treatment course of attentional deficits and overall there is limited availability of cognitive training interventions as a method of treating attentional limitations. Those that exist are frequently delivered on laptops or iPads, do not take into consideration the following because of the devices used:

Detailed behavioral pattern prior to and following response cannot be monitored.

Data collection only takes place on one device, instead of three or more.

Testing attention in response to various degrees of spatial differentiation is not possible.

The deficiency for data-driven methods which aid in diagnosing and treating these and other disorders, a need exists for a platform which that collects, stores, and analyzes data to reveal patterns in physical and cognitive behaviors.

Further, engaging those with ADHD, ASD and other conditions in their treatment is another barrier to recovery for which an evidence-based tool is necessary. Research shows that games appeal to this barrier and provide this engagement.

In the prior applications and patents referenced above, particularly U.S. Pat. No. 9,014,614, we describe a gaming platform that employ a number of movable cubes at communicate with a computer. The platform collects data that was previously unavailable in nature and in quantity. The platform captures response and inter-response movement patterns from each player. The system then combines this data with demographic information and analyzes it through to produce a profile that explains player behavior.

The data is produced by hands-on game interactions and responses. This platform which employs a gaming system improves engagement by taking advantage of psychological predisposition of humans to entertaining challenges triggered by visual and auditory stimulus. The techniques applied to this new type of data make it possible to evaluate and treat attention deficits and/or many of their comorbidities such as depression, anxiety, and ASD.

This platform employs a tangible-graphical user interface. One type of such device, illustrated in FIG. 1 of U.S. Pat. No. 9,014,614, is known as “Sifteo Cubes,” a game platform made by Sifteo Inc. of San Francisco, Calif. Sifteo Cubes are motion-sensitive wireless blocks, which contain multiple sensors and variable displays. They can interact with each other on all four sides. They can be manipulated by hand.

Because of the features of such devices, this game system allows for a third dimension to be studied, a limitation of traditional hardware devices. For example, computers cannot indicate what the participant is doing between disturbances. In contrast, these devices can offer clues about movement in the tilting, pressing, and neighboring actions. This is at a very granular level, because each device has an accelerometer. Further, the gaming platform is designed to capture data at a sub-second second interval. Additionally, while an iPad might offer clues about participant behavior between disturbances, the platform itself is less interactive because in-game interactions do not require tangible movements.

A further advantage of using this system is the insight into spatial attention capabilities. Neither computer-based tests nor the iPad can test auditory spatial attention and visual spatial attention. The platform captures this type of data, creating a more robust tool.

Some other advantages of this platform versus existing technology like iPad/iTouch/iPhone include:

Behavioral pattern prior to and following response can be monitored.

Eliminates effect of reading disability on reaction time and patterns of response.

Multiple devices allows for granular data to be gathered during test session.

Platform provides auditory patterns of response.

Testing for auditory spatial attention and visual spatial attention.

The platform us broadly useful for gathering data that is useful for diagnosing issues in all cognitive areas. This game or test is designed to test several domains of attention by including a combination of multi-task approaches while monitoring behavior prior and following a response by analyzing the degree of movement of the devices as difficulty of the game increases including adding a spatial disturbance in the later stages of the game. In addition to providing diagnostics for executive functioning deficits, this tool can treat these deficits by analyzing the pattern of manipulation of the tangible-graphical device using game based applications, giving instant feedback to fine tune their abilities. This feedback, over time, is a behavioral based intervention that can improve attentional capabilities as a measure of executive functioning without requiring medications. It can also monitor medication treatment effects over time.

Physically active games influence dopamine and norepinephrine, chemicals in the brain that regulate attention, mood, memory and learning. Data is captured real time in response to game play and stored. Thereafter, the captured data is parsed and accumulated using inventive algorithms into predictive data that can be used by a practitioner in diagnosing or treating cognitive disorders. The present invention describes an improved gaming surface that is useful in gaming platforms as described in U.S. Pat. No. 9,014,614

### SUMMARY OF THE INVENTION

This novel gaming surface employs elements disposed on a surface, where the elements embody a physical or digital form. These elements represent positions for controlling rules, and consequently the player/participant, during game/test situations. Specifically the pattern existing in this physical or digital space is used to stretch the attentional and focus capabilities of the user, resulting in a stimulation/aggravation of key neurological functions. These functions are important when assessing and treating neurological conditions and overall states. This allows the creation of a very detailed behavioral profile, which is then captured from the surface through digital mechanisms or computational blocks that capture data.

### BRIEF DESCRIPTION OF THE DRAWING

FIG. 1 illustrates an arrangement of surface elements.

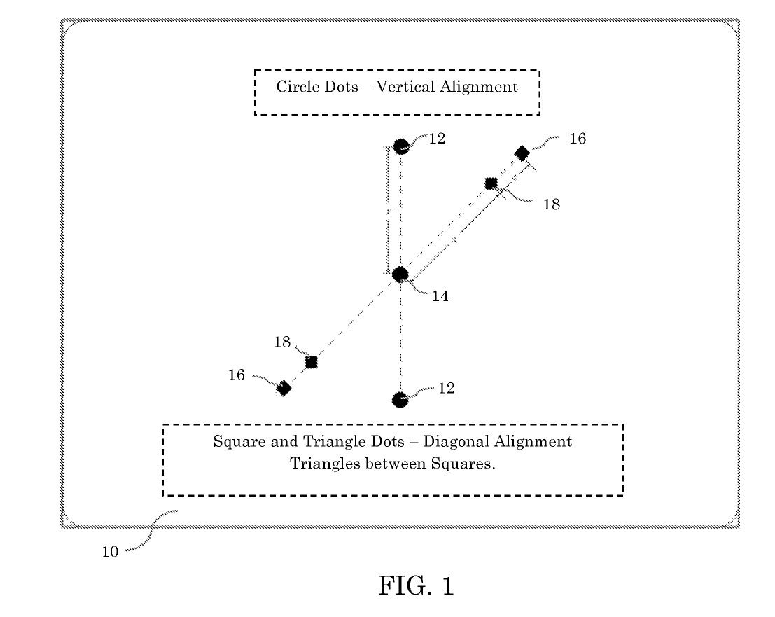

### DETAILED DESCRIPTION

The novel gaming surface 10 has the following properties. The important elements force positioning of the users attention to vertical or diagonal arrangements, forcing stimulation of key brain functions. The elements on the surface can exist digitally, or, with computational blocks (anything from wooden blocks to digital cubes on the game board may equivalently be employed) that can be moved to the position of the elements. In this way, information is always collected about the behavior that corresponds to the movement in and around that specific position. The rules or play of the game/test direct the player/participant to move and/or focus themselves to one of the specified areas/elements of the surface. The configuration of the elements is need to assess visuospatial attention capabilities by requiring the participant to move objects in a specific configuration, while doing this is a controlled, predetermined fashioned which can be studied in clinical trials and treatment settings.

The surface 10 preferably is 14 inches×16 inches; however this is merely an example of possible sizing

Circle: each remote circle dot 12 is vertically 3 inches apart from center (Circle) dot 14, above and below.

Square: each square dot 16 is diagonally spaced 3 inches out from center (Circle) dot 14. This is the first ‘outer’ ring of dots from the center location.

Diamond: each diamond dot 18 is diagonally spaced 4 inches out from center (Circle) dot. It is important that this is stretched far out from the center to really force the spatial distracter.

The placement of these dots allows for a controlled sequence of actions, known as a game, with the game blocks to be monitored, tracked, and analyzed. The actions performed during this sequence, and the speed, movement, and accuracy in how the player responds with the blocks indicates key behavioral characteristics associated with attention.

The game board provides a boundary for actions, controlling responses to a space that can be measured. The arrangement of dots is strategic in parsing out attention patterns. For example, the vertical arrangement of dots is considered the baseline, where the players attention is strained the least. A game piece such as a digital cube or other apparatus can be placed on these 3 dots, which are arranged 2 inches apart, in a vertical line. These dots are all within a normal field of vision, meaning they will not need to move their head or eyes to maintain the objects in site, and assess a player's ability to focus on objects in a vertical dimension. The next level of complexity is spacing the dots out diagonally, 2 inches apart. This configuration of dots is not a common spacing pattern (horizontal or vertically) and thus can act as a distraction to the player, this increasing the complexity of the task, but all dots continue to be in one field of vision. The last configuration of dots is spacing the dots 3 inches apart in a diagonal pattern. This places the dots outside the normal field of vision, and thus stresses the player's ability to maintain focus on all three dots at the same time, while also needing to also contend with the distraction of having objects in a diagonal configuration. There are two sets of diagonal dots, the dots slanted down to the right is for right handed players and the dots slanted down to the left is for left handed players. The reason for the two sets is so players' arm movements will not be hampered by the lower dots based on the slant of the diagonal assigned as above. All of these configurations are important to test different aspects of a player's visuospatial attention capability, and can be challenged in therapeutic way. The best method to test attention is to create a distraction and then measure the response and/or ability to concentrate despite it exists.

The sequence of actions can be directed by the blocks themselves if they are intelligent or contain a mechanism to interact with each other. One embodiment is the Sifteo Cubes, which are tangible digital blocks that interact with each other. The blocks were programmed with a Go/No Go Task, called Groundskeeper, described in detail in incorporated U.S. Pat. No. 9,014,614.

The player is first read this script regarding the game:

Today you will be playing a game called Groundskeeper. It will be used to measure your attention. The goal is to move the mallet cube to the left or right side of the cube showing an image of a gopher. When you have a correct hit, you will hear a ‘boink’ noise. There will be no noise for an incorrect hit. Keep in mind you only want to hit the gopher! Do not let other images, like birds or rabbits, or noises distract you from this task.

There will be a short pause between each level. The first level that you play is for practice and will not be counted against your final score. After each level, a voice will instruct you to move the cubes to colored dots on the game board. Note that the mallet cube should not be moved to these dots.

BE SURE TO USE TWO HANDS AND HOLD THE CUBES TOGETHER UNTIL YOU HEAR A SOUND. IF YOU DO NOT HEAR A BOINK SOUND, YOU DO NOT GET A POINT. MOVE AS QUICKLY AS POSSIBLE!

The game then starts, played entirely from the blocks on the game board. Each level or session of the game follows these parameters:

Groundskeeper Sequence

Each session is 1.5 minutes with 20 seconds between sessions. There will be a ten second countdown on the screen prior to each session. Program will automatically progress to the next session without intervention by person conducting the test. Total run time is 24 min (plus 5 min for pauses).

Session 1: Screen shot of gopher, groundskeeper or grass (neutral) presented for 1, 2 or 3 seconds at a random frequency. Cubes placed vertically, 2 inches apart.
Go/no go task

Session 2: Gopher, groundskeeper or grass screenshot with visual disturbance at a low degree (one bird showing up on screen) alternating with screenshot of grass presented for 1, 2 or 3 seconds at random frequency. Cubes placed vertically, 2 inches apart.
Visual disturbance, low frequency

Session 3: Gopher, groundskeeper or grass screenshot with visual disturbance at a high degree (bird and rabbit) alternating with screenshot of grass presented for 1, 2 or 3 seconds at random frequency. Cubes placed vertically, 2 inches apart.
Visual disturbance, high frequency

Session 4: Screen shot of gopher, groundskeeper or grass screenshot presented for 1, 2 or 3 seconds at a random frequency with auditory disturbance at a low degree (one bird chirping) occurring at random frequencies for 1, 2 or 3 seconds, not in concert with screen shot frequency. Cubes placed vertically, 2 inches apart.
Auditory disturbance, low frequency

Session 5: Screen shot of gopher, groundskeeper or grass screenshot presented for 1, 2 or 3 seconds at a random frequency with auditory disturbance at a high degree (multiple birds chirping) occurring at random frequencies for 1, 2 or 3 seconds, not in concert with screen shot frequency. Cubes placed vertically, 2 inches apart.
Auditory disturbance, high frequency

Session 6: Gopher, groundskeeper or grass screenshot with visual and auditory disturbances at a low degree (one bird chirping) occurring at random frequencies for 1, 2 or 3 seconds. Cubes placed vertically, 2 inches apart.
Visual and auditory disturbance, low frequency

Session 7: Gopher, groundskeeper or grass screenshot with visual and auditory disturbances at a high degree (bird and rabbit and chirping) occurring at random frequencies for 1, 2 or 3 seconds. Cubes placed vertically, 2 inches apart.
Visual and auditory disturbance, high frequency

Session 8: Gopher, groundskeeper or grass screenshot with spatial disturbance at a low degree. Cube set diagonally. Spaced at 2 inches apart and occurring at random frequencies for 1, 2 or 3 seconds.
Spatial disturbance, low frequency

Session 9: Gopher, groundskeeper or grass screenshot with spatial disturbance at a low degree combined with low frequency visual disturbance. Cube set diagonally. Spaced at 2 inches apart and occurring at random frequencies for 1, 2 or 3 seconds.
Spatial disturbance, low frequency, low frequency visual disturbance

Session 10: Gopher, groundskeeper or grass screenshot with spatial disturbance at a low degree combined with low frequency auditory disturbance. Cube set diagonally. Spaced at 2 inches apart and occurring at random frequencies for 1, 2 or 3 seconds.
Spatial disturbance, low frequency, low frequency auditory disturbance

Session 11: Gopher, groundskeeper or grass screenshot with spatial disturbance at a low degree combined with low frequency visual and auditory disturbance. Cube set diagonally. Spaced at 2 inches apart and occurring at random frequencies for 1, 2 or 3 seconds.
Spatial disturbance, low frequency, low frequency visual and auditory disturbance

Session 12: Gopher, groundskeeper or grass screenshot with spatial disturbance at a low degree. Cube set diagonally. Spaced at 3 inches apart and occurring at random frequencies for 1, 2 or 3 seconds.
Spatial disturbance, high frequency

Session 13: Gopher, groundskeeper or grass screenshot with spatial disturbance at a high degree combined with low frequency visual disturbance. Cube set diagonally. Spaced at 3 inches apart and occurring at random frequencies for 1, 2 or 3 seconds.
Spatial disturbance, high frequency, low frequency visual disturbance

Session 14: Gopher, groundskeeper or grass screenshot with spatial disturbance at a high degree combined with low frequency auditor disturbance. Cube set diagonally. Spaced at 3 inches apart and occurring at random frequencies for 1, 2 or 3 seconds.
Spatial disturbance, high frequency, low frequency auditory disturbance

Session 15: Gopher, groundskeeper or grass screenshot with spatial disturbance at a high degree combined with low frequency visual and auditory disturbance. Cube set diagonally. Spaced at 3 inches apart and occurring at random frequencies for 1, 2 or 3 seconds.
Spatial disturbance, high frequency, low frequency visual and auditory disturbance

Session 16: Screen shot of gopher, groundskeeper or grass (neutral) presented for 1, 2 or 3 seconds at a random frequency.
Go/no go task with learning curve.

Once the game is complete, the data recorded regarding player performance, which includes movement, reaction, correct, incorrect, and omissions is wirelessly transmitted to the cloud for analysis. CogCubed has developed algorithms to make sense of this information which has been published and is described in U.S. Pat. No. 9,014,614. The response differences between spatial and non-spatial distractions is a key element in understanding conditions associated with attention like ADHD, Anxiety, Depression, and Autism.

The description of the various embodiments above are by example are not intended to limit the scope of the appended claims. Furthermore, unless the claims specifically use “means for” or “step for” in a claim or a claim element, applicant specifically intends that the claim or claim element as the case not be construed under 35 USC §112(f).

We claim:

1. An apparatus, comprising;
a rectangular game board having demarked thereon in two sets of marks, each set forming a line, wherein a first line is orientated in a first direction parallel with a side of the rectangular game board, and a second line orientated in a second direction diagonal to the first line.

2. The apparatus according to claim 2, wherein the first line has three marks, one a centered mark.

3. The apparatus according to claim 1, wherein the second line has five marks, one centered mark.

4. The apparatus according to claim 3, wherein the five marks comprise two end marks, and two median marks spaced between the end marks and the centered mark.

5. The apparatus according to claim 4, wherein two median marks are spaced two thirds the distance from the centered mark and a respective end mark.

6. A method comprising:
employing a gaming platform to position one or more objective cubes having an alterable display on one or more marks disposed on gaming board and a mallet cube on a centered mark; and thereafter asking the player to perform a task consisting of moving the mallet cube to hit an objective cube according to whether the objective cube has a predefined screenshot displayed; wherein the gaming board comprises a rectangular game board having demarked thereon in two sets of marks, each set forming a line; wherein a first line is orientated in a first direction parallel with a side of the rectangular game board, and a second line orientated in a second direction diagonal to the first line.

7. The method according to claim 6, wherein the first line has three marks, one a centered mark.

8. The method according to claim 6, wherein the second line has five marks, one centered mark.

9. The method according to claim 8, wherein the five marks comprise two end marks, and two median marks spaced between the end marks and the centered mark.

10. The method according to claim 9, wherein two median marks are spaced two thirds the distance from the centered mark and a respective end mark.

11. The method according to claim 6, further comprising gathering data from the response of the player to performance of one or more of tasks where tasks are varied by locating the one or more objective cubes on different marks and providing different distractions.

12. The method according to claim 11, further comprising diagnosing and treating a patient for neurological disorders in accordance with the data analysis.
